(19) United States
(12) Patent Application Publication
(10) Pub. No.: US 2009/0253714 A1
(43) Pub. Date: Oct. 8, 2009

(54) Methods of reducing risk of infection from pathogens

(76) Inventors: Michael R. Johnson, Chapel Hill, NC (US); Samuel E. Hopkins, Raleigh, NC (US)

Correspondence Address: BRINKS, HOFER, GILSON & LIONE, P.O. BOX 1340, MORRISVILLE, NC, 27560, US

(21) Appl. No.: 10/920,527

(22) Filed: Aug. 18, 2004

Related U.S. Application Data
(60) Provisional application No. 60/496,517, filed on Aug. 20, 2003.

(51) Int. Cl. A61K 31/4965; A61L 9/04

(52) U.S. Cl. 514/255.05; 514/255.06

(57) ABSTRACT

Prophylactic treatment methods are provided for protection of individuals and/or populations against infection from airborne pathogens. In particular, prophylactic treatment methods are provided comprising administering amiloride, benzamil, phenamil or pharmaceutically acceptable salts thereof to one or more members of a population at risk of exposure to or already exposed to one or more airborne pathogens, either from natural sources or from intentional release of pathogens into the environment.

### CROSS REFERENCE TO RELATED APPLICATIONS

This application claims the benefit of U.S. Provisional Application No. 60/496,517, filed Aug. 20, 2003, incorporated herein by reference in its entirety.

### BACKGROUND OF THE INVENTION

1. Field of the Invention

The present invention relates to the use of pyrazinoylguanidines such as amiloride, benzamil or phenamil for prophylactic, post-exposure prophylactic, or preventive treatment against diseases or conditions caused by pathogens, particularly pathogens which may be used in bioterrorism.

2. Description of the Related Art

In recent years, a variety of research programs and biodefense measures have been put into place to deal with concerns about the use of biological agents in acts of terrorism. These measures are intended to address concerns regarding bioterrorism or the use of microorganisms or biological toxins to kill people, spread fear, and disrupt society. For example, the National Institute of Allergy and Infectious Diseases (NIAID) has developed a Strategic Plan for Biodefense Research which outlines plans for addressing research needs in the broad area of bioterrorism and emerging and reemerging infectious diseases. According to the plan, the deliberate exposure of the civilian population of the United States to Bacillus anthracis spores revealed a gap in the nation's overall preparedness against bioterrorism. Moreover, the report details that these attacks uncovered an unmet need for tests to rapidly diagnose, vaccines and immunotherapies to prevent, and drugs and biologics to cure disease caused by agents of bioterrorism.

Much of the focus of the various research efforts has been directed to studying the biology of the pathogens identified as potentially dangerous as bioterrorism agents, studying the host response against such agents, developing vaccines against infectious diseases, evaluating the therapeutics currently available and under investigation against such agents, and developing diagnostics to identify signs and symptoms of threatening agents. Such efforts are laudable but, given the large number of pathogens which have been identified as potentially available for bioterrorism, these efforts have not yet been able to provide satisfactory responses for all possible bioterrorism threats. Additionally, many of the pathogens identified as potentially dangerous as agents of bioterrorism do not provide adequate economic incentives for the development of therapeutic or preventive measures by industry. Moreover, even if preventive measures such as vaccines were available for each pathogen which may be used in bioterrorism, the cost of administering all such vaccines to the general population is prohibitive.

Until convenient and effective treatments are available against every bioterrorism threat, there exists a strong need for preventative or prophylactic treatments which can prevent or reduce the risk of infection from pathogenic agents.

### BRIEF SUMMARY OF THE INVENTION

The present invention provides such methods of prophylactic treatment. In one aspect of the invention, a prophylactic treatment method is provided comprising administering a prophylactically effective amount of amiloride, benzamil, phenamil or a pharmaceutically acceptable salt thereof to an individual in need of prophylactic treatment against infection from one or more airborne pathogens.

In another aspect, a prophylactic treatment method is provided for reducing the risk of infection from an airborne pathogen which can cause a disease in a human, said method comprising administering an effective amount of amiloride, benzamil, phenamil or a pharmaceutically acceptable salt thereof to the lungs of the human who may be at risk of infection from the airborne pathogen but is asymptomatic for the disease, wherein the effective amount of amiloride, benzamil, phenamil or a pharmaceutically acceptable salt is sufficient to reduce the risk of infection in the human.

### DETAILED DESCRIPTION OF THE INVENTION

The prophylactic treatment methods of the present invention may be used in situations where a segment of the population has been, or is believed to have been, exposed to one or more airborne pathogens. The prophylactic treatment methods may additionally be used in situations of ongoing risk of exposure to or infection from airborne pathogens. Such situations may arise due to naturally occurring pathogens or may arise due to a bioterrorism event wherein a segment of the population is intentionally exposed to one or more pathogens. The individuals or portion of the population believed to be at risk from infection can be treated according to the methods disclosed herein. Such treatment preferably will commence at the earliest possible time, either prior to exposure if imminent exposure to a pathogen is anticipated or possible or after the actual or suspected exposure. Typically, the prophylactic treatment methods will be used on humans asymptomatic for the disease for which the human is believed to be at risk. The term “asymptomatic” as used herein means not exhibiting medically recognized symptoms of the disease, not yet suffering from infection or disease from exposure to the airborne pathogens, or not yet testing positive for a disease.

Many of the pathogenic agents identified by NIAID have been or are capable of being aerosolized such that they may enter the body through the mouth or nose, moving into the bodily airways and lungs. These areas of the body have mucosal surfaces which naturally serve, in part, to defend against foreign agents entering the body. The mucosal surfaces at the interface between the environment and the body have evolved a number of “innate defense”, i.e., protective mechanisms. A principal form of such innate defense is to cleanse these surfaces with liquid. Typically, the quantity of the liquid layer on a mucosal surface reflects the balance between epithelial liquid secretion, often reflecting anion (Cl⁻ and/or HCO₃⁻) secretion coupled with water (and a cation counter-ion), and epithelial liquid absorption, often reflecting Na⁺ absorption, coupled with water and counter anion (Cl⁻ and/or HCO₃⁻).

R. C. Boucher, in U.S. Pat. No. 6,264,975, describes methods of hydrating mucosal surfaces, particularly nasal airway surfaces, by administration of pyrazinoylguanidine sodium channel blockers. These compounds, typified by amiloride, benzamil and phenamil, are effective for hydration of the mucosal surfaces. U.S. Pat. No. 5,656,256, describes methods of hydrating mucous secretions in the lungs by administration of benzamil or phenamil, for example, to treat diseases such as cystic fibrosis and chronic bronchitis. U.S. Pat. No. 5,725,842 is directed to methods of removing retained mucus secretions from the lungs by administration of amiloride.

It has now been discovered that amiloride, benzamil and phenamil may be used in prophylactic treatment methods to protect humans in whole or in part, against the risk of infection from pathogens which may or may not have been purposely introduced into the environment, typically into the air, of a populated area. Such treatment may be effectively used to protect those who may have been exposed where a vaccine is not available or has not been provided to the population exposed and/or in situations where treatments for the infection resulting from the pathogen to which a population has been subjected are insufficient or unavailable altogether.

Without being bound by any theory, it is believed that the amiloride, benzamil and/or phenamil surprisingly may be used on substantially normal or healthy lung tissue to prevent or reduce the uptake of airborne pathogens and/or to clear the lungs of all or at least a portion of such pathogens. Preferably, the amiloride, benzamil and/or phenamil will prevent or reduce the viral or bacterial uptake of airborne pathogens. The ability of amiloride, benzamil and/or phenamil to hydrate mucosal surfaces is believed to function to first hydrate lung mucous secretions, including mucous containing the airborne pathogens to which the human has been subjected, and then facilitate the removal of the lung mucous secretions from the body. By functioning to remove the lung mucous secretions from the body, the amiloride, benzamil and/or phenamil thus prevents or, at least, reduces the risk of infection from the pathogen(s) inhaled or brought into the body through a bodily airway.

The present invention is concerned primarily with the prophylactic, post exposure, rescue and therapeutic treatment of human subjects, but may also be employed for the treatment of other mammalian subjects, such as dogs and cats, for veterinary purposes, and to the extent the mammals are at risk of infection or disease from airborne pathogens.

The term “airway” as used herein refers to all airways in the respiratory system such as those accessible from the mouth or nose, including below the larynx and in the lungs, as well as air passages in the head, including the sinuses, in the region above the larynx.

The terms “pathogen” and “pathogenic agent” are interchangeable and, as used herein, means any agent that can cause disease or a toxic substance produced by a pathogen that causes disease. Typically, the pathogenic agent will be a living organism that can cause disease. By way of example, a pathogen may be any microorganism such as bacterium, protozoan or virus that can cause disease.

The term “airborne pathogen” means any pathogen which is capable of being transmitted through the air and includes pathogens which travel through air by way of a carrier material and pathogens either artificially aerosolized or naturally occurring in the air.

The term “prophylactic” as used herein means the prevention of infection, the delay of infection, the inhibition of infection and/or the reduction of the risk of infection from pathogens, and includes pre- and post-exposure to pathogens. The prophylactic effect may, inter alia, involve a reduction in the ability of pathogens to enter the body, or may involve the removal of all or a portion of pathogens which reach airways and airway surfaces in the body from the body prior to the pathogens initiating or causing infection or disease. The airways from which pathogens may be removed, in whole or part, include all bodily airways and airway surfaces with mucosal surfaces, including airway surfaces in the lungs.

The compounds useful in the present invention may include pyrazinoylguanidines as described in U.S. Pat. No. 3,313,813, to E. Cragoe, incorporated herein by reference in its entirety, particularly amiloride, benzamil and phenamil. Amiloride is 3,5-diamino-N-(aminoiminomethyl)-6-chloropyrazinecarboxamide. Benzamil is 3,5-diamino-6-chloro-N-(benzylaminoaminomethylene)pyrazinecarboxamide and phenamil is 3,5-diamino-6-chloro-N-(phenylaminoaminomethylene)pyrazinecarboxamide. Each of these compounds is disclosed in U.S. Pat. No. 3,313,813. Amiloride, benzamil and phenamil may be prepared by the procedures described in U.S. Pat. No. 3,313,813, in combination with procedures known to those skilled in the art.

The terms “amiloride”, “benzamil” and “phenamil” as used herein, include the pharmaceutically acceptable salts thereof. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine.

Amiloride, benzamil or phenamil used to prepare compositions for the present invention may alternatively be in the form of a pharmaceutically acceptable free base of amiloride or benzamil or phenamil. Because the free base of the compound is less soluble than the salt, free base compositions are employed to provide more sustained release of the compound to the lungs. Amiloride, benzamil or phenamil present in the lungs in particulate form which has not gone into solution is not available to induce a physiological response, but serves as a depot of bioavailable drug which gradually goes into solution.

The active compounds disclosed herein may be administered to the lungs of a patient by any suitable means, but are preferably administered by administering an aerosol suspension of respirable particles comprised of the active compound, which the subject inhales. The compounds may be inhaled through the mouth or the nose. The active compound can be aerosolized in a variety of forms, such as, but not limited to, dry powder inhalants, metered dose inhalants or liquid/liquid suspensions. The respirable particles may be liquid or solid. The quantity of amiloride, benzamil or phenamil included may be an amount sufficient to achieve dissolved concentrations of amiloride, benzamil or phenamil on the airway surfaces of the subject of from about 10⁻⁷ to about 10⁻³ moles/liter, and more preferably from about 10⁻⁶ to about 10⁻⁴ moles/liter.

In one aspect of the invention, the particulate amiloride, benzamil or phenamil composition may contain both a free base of amiloride, phenamil or benzamil and a pharmaceutically acceptable salt such as amiloride hydrochloride, benzamil hydrochloride or phenamil hydrochloride to provide both early release of and sustained release of amiloride, benzamil or phenamil for dissolution into the mucous secretions of the lungs. Such a composition serves to provide both early relief to the patient, and sustained relief over time. Sustained relief, by decreasing the number of daily administrations required, is expected to increase patient compliance with a course of amiloride or benzamil or phenamil treatments.

Solid or liquid particulate amiloride, benzamil or phenamil prepared for practicing the present invention should include particles of respirable size: that is, particles of a size sufficiently small to pass through the mouth and larynx upon inhalation and into the bronchi and alveoli of the lungs. In general, particles ranging from about 1 to 5 microns in size (more particularly, less than about 4.7 microns in size) are respirable. Particles of non-respirable size which are included in the aerosol tend to be deposited in the throat and swallowed, and the quantity of non-respirable particles in the aerosol is preferably minimized. For nasal administration, a particle size in the range of 10-500 μm is preferred to ensure retention in the nasal cavity. Nasal administration may be useful where the pathogen typically enters through the nose. However, it is preferred to administer at least a portion of the amiloride, benzamil or phenamil in a dosage form which reaches the lungs to ensure effective prophylactic treatment in cases where the pathogen is expected to reach the lungs.

The dosage of active compound will vary depending on the prophylactic effect desired and the state of the subject, but generally may be an amount sufficient to achieve dissolved concentrations of active compound on the airway surfaces of the subject of from about 10⁻⁷ to about 10⁻³ moles/liter, and more preferably from about 10⁻⁶ to about 10⁻⁴ moles/liter. Depending upon the solubility of the particular formulation of active compound administered, the daily dose may be divided among one or several unit dose administrations. The daily dose by weight may range from about 1 to about 20 milligrams of respirable amiloride, benzamil or phenamil particles for a human subject, depending upon the age and condition of the subject, preferably a daily dose of about 1 to about 10 mg. A currently preferred unit dose is about 2 milligrams of respirable amiloride, benzamil or phenamil particles given at a regimen of four administrations per day. The dosage may be provided as a prepackaged unit by any suitable means (e.g., encapsulating in a gelatin capsule).

Pharmaceutical formulations suitable for airway administration include formulations of solutions, emulsions, suspensions and extracts. See generally, J. Naim, Solutions, Emulsions, Suspensions and Extracts, in Remington: The Science and practice of Pharmacy, chap. 86 (19^{th} ed. 1995). Pharmaceutical formulations suitable for nasal administration may be prepared as described in U.S. Pat. No. 4,389,393 to Schor; U.S. Pat. No. 5,707,644 to Ilium, U.S. Pat. No. 4,294,829 to Suzuki, and U.S. Pat. No. 4,835,142 to Suzuki.

In the manufacture of a formulation according to the invention, active agents or the physiologically acceptable salts or free bases thereof are typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a capsule, which may contain from 0.5% to 99% by weight of the active compound. One or more active compounds may be incorporated in the formulations of the invention, which formulations may be prepared by any of the well-known techniques of pharmacy consisting essentially of admixing the components.

Aerosols or mists of liquid particles comprising the active compound may be produced by any suitable means, such as, for nasal administration, by a simple nasal spray with the active compound in an aqueous pharmaceutically acceptable carrier such as sterile saline solution or sterile water. Other means include producing aerosols with a pressure-driven aerosol nebulizer or an ultrasonic nebulizer. See, e.g., U.S. Pat. No. 4,501,729. Nebulizers are commercially available devices which transform solutions or suspensions of the active ingredient into a therapeutic aerosol mist either by means of acceleration of compressed gas, typically air or oxygen, through a narrow venturi orifice or by means of ultrasonic agitation. Suitable formulations for use in nebulizers consist of the active ingredient in a liquid carrier, the active ingredient comprising up to 40% w/w of the formulation, but preferably less than 20% w/w. The carrier is typically water (and most preferably sterile, pyrogen-free water) or a dilute aqueous alcoholic solution, preferably made isotonic with body fluids by the addition of, for example, sodium chloride.

Aerosols or mists of solid particles comprising the active compound may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. Such aerosol generators are known in the art. By way of example, see U.S. Pat. No. 5,725,842.

One illustrative type of solid particulate aerosol generator is an insulator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the active ingredient or of a powder blend comprising the active ingredient, a suitable powder diluent, such as lactose, and an optional surfactant. The active ingredient typically comprises from 0.1 to 100 w/w of the formulation.

A second type of illustrative aerosol generator comprises a metered dose inhaler. Metered dose inhalers are pressurized aerosol dispensers, typically containing a suspension or solution formulation of the active ingredient in a liquefied propellant. During use these devices discharge the formulation through a valve adapted to deliver a metered volume, typically from 10 to 150 μl to produce a fine particle spray containing the active ingredient. Any propellant may be used in carrying out the present invention, including both chlorofluorocarbon-containing propellants and non-chlorofluorocarbon-containing propellants. Suitable propellants include certain chlorofluorocarbon compounds, for example, dichlorodifluoromethane, trichlorofluoromethane, dichlorotetrafluoroethane and mixtures thereof.

The formulation may additionally contain one or more co-solvents, for example, ethanol, surfactants, such as oleic acid or sorbitan trioleate, antioxidants, preservatives such as methyl hydroxybenzoate, volatile oils, buffering agents and suitable flavoring agents.

Compositions containing respirable dry particles of micronized amiloride, benzamil or phenamil may be prepared by grinding the dry amiloride, phenamil or benzamil with a mortar and pestle, and then passing the micronized composition through a 400 mesh screen to break up or separate out large agglomerates. The active compound may be formulated alone (i.e., the solid particulate composition may consist essentially of the active compound) or in combination with a dispersant, diluent or carrier, such as sugars (i.e., lactose, sucrose, trehalose, mannitol) or other acceptable excipients for lung or airway delivery, which may be blended with the active compound in any suitable ratio (e.g., a 1 to 1 ratio by weight). The dry powder solid particulate compound may be obtained by methods known in the art, such as spray-drying, milling, freeze-drying, and the like.

The aerosol or mist, whether formed from solid or liquid particles, may be produced by the aerosol generator at a rate of from about 10 to about 150 liters per minute, more preferably from about 30 to about 150 liters per minute, and most preferably about 60 liters per minute. Aerosols containing greater amounts of medicament may be administered more rapidly.

Other medicaments may be administered with the active compounds disclosed if such medicament is compatible with the active compound and other ingredients in the formulation and can be administered as described herein.

The pathogens which may be protected against by the prophylactic post exposure, rescue and therapeutic treatment methods of the invention include any pathogens which may enter the body through the mouth, nose or nasal airways, thus proceeding into the lungs. Typically, the pathogens will be airborne pathogens, either naturally occurring or by aerosolization. The pathogens may be naturally occurring or may have been introduced into the environment intentionally after aerosolization or other method of introducing the pathogens into the environment. Many pathogens which are not naturally transmitted in the air have been or may be aerosolized for use in bioterrorism.

The pathogens for which the treatment of the invention may be useful includes, but is not limited to, category A, B and C priority pathogens as set forth by the NIAID. These categories correspond generally to the lists compiled by the Centers for Disease Control and Prevention (CDC). As set up by the CDC, Category A agents are those that can be easily disseminated or transmitted person-to-person, cause high mortality, with potential for major public health impact. Category B agents are next in priority and include those that are moderately easy to disseminate and cause moderate morbidity and low mortality. Category C consists of emerging pathogens that could be engineered for mass dissemination in the future because of their availability, ease of production and dissemination and potential for high morbidity and mortality.

Category A:
Bacillus anthracis (anthrax),
Clostridium botulinum (botulism),
Yersinia pestis (plague),
Variola major (smallpox) and other pox viruses,
Francisella tularensis (tularemia),
Viral hemorrhagic fevers
Arenaviruses,
   LCM (lymphocytic choriomeningitis), Junin virus,
   Machupo virus, Guanarite virus,
   Lassa Fever,
Bunyaviruses,
   Hantavirus,
   Rift Valley Fever,
Flaviviruses,
   Dengue,
Filoviruses,
   Ebola
   Marburg;

Category B:
Burkholderia pseudomallei (melioidosis),
Coxiella burnetii (Q fever),
Brucella species (brucellosis),
Burkholderia mallei (glanders),
Ricin toxin from Ricinus communes,
Epsilon toxin of Clostridium perfringens,
Staphylococcal enterotoxin B,
Typhus fever (Rickettsia prowazekii),
Food and water-borne pathogens
   bacteria:
      Diarrheagenic Escherichia coli,
      Pathogenic vibrios,
      Shigella species,
      Salmonella species,
      Listeria monocytogenes,
      campylobacter jejuni,
      Yersinia enterocolitica;
   Viruses
      Caliciviruses,
      Hepatitis A;
   Protozoa
      Cryptosporidium parvum,
      Cyclospora cayatenensis,
      Giardia lamblia,
      Entamoeba histolytica,
      Toxoplasma,
      Microsporidia, and
   Additional viral encephalitides
      West Nile virus,
      LaCrosse,
      California encephalitis,
      Venezuelan equine encephalitis,
      Eastern equine encephalitis,
      Western equine encephalitis,
      Japanese encephalitis virus and
      Kyasanur forest virus, and

Category C: emerging infectious disease threats such as Nipah virus and additional hantaviruses, tickborne hemorrhagic fever viruses such as Crimean Congo hemorrhagic fever virus, tickborne encephalitis viruses, yellow fever, multi-drug resistant tuberculosis, influenza, other rickettsias and rabies.

Additional pathogens which may be protected against or the infection risk therefrom reduced include influenza viruses, rhinoviruses, adenoviruses and respiratory syncytial viruses, and the like. A further pathogen which may be protected against is the coronavirus which is believed to cause severe acute respiratory syndrome (SARS).

A number of the above-listed pathogens are known to be particularly harmful when introduced into the body through the air. For example, Bacillus anthracis, the agent which causes anthrax, has three major clinical forms, cutaneous, inhalational, and gastrointestinal. All three forms may lead to death but early antibiotic treatment of cutaneous and gastrointestinal anthrax usually cures those forms of anthrax. Inhalational anthrax, on the other hand, is a potentially fatal disease even with antibiotic treatment. Initial symptoms may resemble a common cold. After several days, the symptoms may progress to severe breathing problems and shock. For naturally occurring or accidental infections, even with appropriate antibiotics and all other available supportive care, the historical fatality rate is believed to be about 75 percent, according to the NIAID. Inhalational anthrax develops after spores are deposited in alveolar spaces and subsequently ingested by pulmonary alveolar macrophages. Surviving spores are then transported to the mediastinal lymph nodes, where they may germinate up to 60 days or longer. After germination, replicating bacteria release toxins that result in disease. This process is interrupted by administration of a prophylactically effective amount of amiloride, benzamil and/or phenamil, as the spores may be wholly or partially eliminated from the body by removal of lung mucous secretions hydrated through the action of the amiloride, benzamil and/or phenamil.

Another pathogen of primary concern as one of the most dangerous potential biological weapons because it is easily transmitted from person to person, no effective therapy exists and few people carry full immunity to the virus, is the small pox virus, Variola major. Smallpox spreads directly from person to person, primarily by aerosolized saliva droplets expelled from an infected person. Initial symptoms include high fever, fatigue, headache and backache followed in two or three days by a characteristic rash.

An embodiment of the present invention provides a method of prophylactically treating one or more individuals exposed or potentially exposed to smallpox virus or other pox virus comprising the administration of a prophylactically effective amount of amiloride, benzamil and/or phenamil. The administration of an effective amount of amiloride, benzamil and/or phenamil will function to allow the Variola major virus or other pox virus present in the aerosolized saliva droplets to which the individual was exposed to be wholly or partially removed from the body by removal of hydrated lung mucous secretions hydrated through the action of amiloride, benzamil and/or phenamil.

The bacterium Yersinia pestis causes plague and is widely available throughout the world. NIAID has reported that infection by inhalation of even small numbers of virulent aerosolized Y. pestis bacilli can lead to pneumonic plague, which has a mortality rate of almost 100% if left untreated. Pneumonic plague has initial symptoms of fever and cough which resemble other respiratory illnesses. Antibiotics are effective against plague but success with antibiotics depends on how quickly drug therapy is started, the dose of inhaled bacteria and the level of supportive care for the patient; an effective vaccine is not widely available.

An embodiment of the present invention provides a method of prophylactically treating one or more individuals exposed or potentially exposed to aerosolized Y. pestis bacilli comprising the administration of amiloride, benzamil and/or phenamil. The administration of an effective amount of amiloride, benzamil and/or phenamil will function to allow the aerosolized Y. pestis bacilli to be wholly or partially removed from the body by removal of hydrated lung mucous secretions hydrated through the action of amiloride, benzamil and/or phenamil.

Botulinum toxin is another substance believed to present a major bioterrorism threat as it is easily released into the environment. Antibiotics are not effective against botulinum toxin and no approved vaccine exists. Although the toxin may be transmitted through food, the botulinum toxin is absorbed across mucosal surfaces and, thus, an embodiment of the present invention provides a method of prophylactically treating one or more individuals exposed or potentially exposed to botulinum toxin comprising the administration of amiloride, benzamil and/or phenamil.

The NIAID has identified the bacteria that causes tularemia as a potential bioterrorist agent because Francisella tularensis is capable of causing infection with as few as ten organisms and due to its ability to be aerosolized. Natural infection occurs after inhalation of airborne particles. Tularemia may be treated with antibiotics and an experimental vaccine exists but knowledge of optimal therapeutic approaches for tularemia is limited because very few investigators are working on this disease. An embodiment of the present invention provides a method of prophylactically treating one or more individuals exposed or potentially exposed to aerosolized Francisella tularensis comprising the administration of amiloride, benzamil and/or phenamil. The administration of an effective amount of amiloride, benzamil and/or phenamil will function to allow the aerosolized Francisella tularensis to be wholly or partially removed from the body by removal of hydrated lung mucous secretions hydrated through the action of amiloride, benzamil and/or phenamil.

The Category B and C bacteria most widely believed to have the potential to infect by the aerosol route include gram negative bacteria such as Brucella species, Burkholderia pseudomallei, Burkholderia mallei, Coxiella burnetii, and select Rickettsia spp. Each of these agents is believed to be capable of causing infections following inhalation of small numbers of organisms. Brucella spp. may cause brucellosis. Four of the six Brucella spp., B. suis, B. melitensis, B. abortus and B. canis, are known to cause brucellosis in humans. Burkholderia pseudomallei may cause melioidosis in humans and other mammals and birds. Burkholderia mallei, is the organism that causes glanders, normally a disease of horses, mules and donkeys but infection following aerosol exposure has been reported, according to NIAID. Coxiella burneti, may cause Q fever and is highly infectious. Infections have been reported through aerosolized bacteria and inhalation of only a few organisms can cause infections. R. prowazekii, R. rickettsii, R. conorrii and R. typhi have been found to have low-dose infectivity via the aerosol route.

An embodiment of the present invention provides a method of prophylactically treating one or more individuals exposed or potentially exposed to aerosolized gram negative bacteria such as Brucella species, Burkholderia pseudomallei, Burkholderia mallei, Coxiella burnetii, and select Rickettsia spp comprising the administration of amiloride, benzamil and/or phenamil. The administration of an effective amount of amiloride, benzamil and/or phenamil will function to allow the aerosolized gram negative bacteria to be wholly or partially removed from the body by removal of hydrated lung mucous secretions hydrated through the action of amiloride, benzamil and/or phenamil.

A number of typically arthropod-borne viruses are believed to pose a significant threat as potential bioterrorist weapons due to their extreme infectivity following aerosolized exposure. These viruses include arboviruses which are important agents of viral encephalitides and hemorrhagic fevers. Such viruses may include alphaviruses such as Venezuelan equine encephalitis virus, eastern equine encephalitis virus and western equine encephalitis virus. Other such viruses may include flaviviruses such as West Nile virus, Japanese encephalitis virus, Kyasanur forest disease virus, tick-borne encephalitis virus complex and yellow fever virus. An additional group of viruses which may pose a threat include bunyaviruses such as California encephalitis virus, or La Crosse virus, Crimean-Congo hemorrhagic fever virus. According to the NIAID, vaccines or effective specific therapeutics are available for only a very few of these viruses. In humans, arbovirus infection is usually initially asymptomatic or causes nonspecific flu-like symptoms such as fever, aches and fatigue.

An embodiment of the present invention provides a method of prophylactically treating one or more individuals exposed or potentially exposed to aerosolized arboviruses comprising the administration of amiloride, benzamil and/or phenamil. The administration of an effective amount of amiloride, benzamil and/or phenamil will function to allow the arboviruses to be wholly or partially removed from the body by removal of hydrated lung mucous secretions hydrated through the action of amiloride, benzamil and/or phenamil.

Certain category B toxins such as ricin toxin from Ricinus communis, epsilon toxin of Clostridium perfringens and Staphylococcal enterotoxin B, also are viewed as potential bioterrorism tools. Each of these toxins may be delivered to the environment or population by inhalational exposure to aerosols. Low dose inhalation of ricin toxin may cause nose and throat congestion and bronchial asthma while higher dose inhalational exposure caused severe pneumonia, acute inflammation and diffuse necrosis of the airways in nonhuman primates. Clostridium perfringens is an anaerobic bacterium that can infect humans and animals. Five types of bacteria exist that produce four major lethal toxins and seven minor toxins, including alpha toxin, associated with gas gangrene, beta toxin, responsible for necrotizing enteritis, and epsilon toxin, a neurotoxin that leads to hemorrhagic enteritis in goats and sheep. Inhalation of Staphylococcus aureus has resulted in extremely high fever, difficulty breathing, chest pain and headache.

An embodiment of the present invention provides a method of prophylactically treating one or more individuals exposed or potentially exposed to aerosolized toxins comprising the administration of amiloride, benzamil and/or phenamil. The administration of an effective amount of amiloride, benzamil and/or phenamil will function to allow the aerosolized toxins to be wholly or partially removed from the body by removal of hydrated lung mucous secretions hydrated through the action of amiloride, benzamil and/or phenamil.

Mycobacterium tuberculosis bacteria causes tuberculosis and is spread by airborne droplets expelled from the lungs when a person with tuberculosis coughs, sneezes or speaks. An embodiment of the present invention provides a method of prophylactically treating one or more individuals exposed or potentially exposed to Mycobacterium tuberculosis bacteria comprising the administration of amiloride, benzamil and/or phenamil. The administration of an effective amount of amiloride, benzamil and/or phenamil will function to allow the Mycobacterium tuberculosis bacteria to be wholly or partially removed from the body by removal of hydrated lung mucous secretions hydrated through the action of amiloride, benzamil and/or phenamil.

The methods of the present invention may also be used against more common pathogens such as influenza viruses, rhinoviruses, adenoviruses and respiratory syncytial viruses (RSV). An embodiment of the present invention provides a method of prophylactically treating one or more individuals exposed or potentially exposed to one of these viruses comprising the administration of amiloride, benzamil and/or phenamil. The administration of an effective amount of amiloride, benzamil and/or phenamil will function to allow the virus to be wholly or partially removed from the body by removal of hydrated lung mucous secretions hydrated through the action of amiloride, benzamil and/or phenamil.

The methods of the present invention may further be used against the virus believed to be responsible for SARS, the coronavirus. Severe acute respiratory syndrome is a respiratory illness that is believed to spread by person-to-person contact, including when someone coughs or sneezes droplets containing the virus onto others or nearby surfaces. The CDC currently believes that it is possible that SARS can be spread more broadly through the air or by other ways that are not currently known. Typically, SARS begins with a fever greater than 100.4° F. Other symptoms include headache and body aches. After two to seven days, SARS patients may develop a dry cough and have trouble breathing.

To the extent SARS is caused by an airborne pathogen, an embodiment of the present invention provides a method of prophylactically treating one or more individuals exposed or potentially exposed to the SARS virus comprising the administration of amiloride, benzamil and/or phenamil. The administration of an effective amount of amiloride, benzamil and/or phenamil will function to allow the virus to be wholly or partially removed from the body by removal of hydrated lung mucous secretions hydrated through the action of amiloride, benzamil and/or phenamil.

The following example illustrates one method for determining the effect of compounds on mucociliary clearance. Amiloride, benzamil and phenamil show positive effects in the assay described.

Pharmacological Effects and Mechanism of Action of the Drug in Animals

The effect of compounds for enhancing mucociliary clearance (MCC) can be measured using an in vivo model described by Sabater et al., Journal of Applied Physiology, 1999, pp. 2191-2196, incorporated herein by reference. A typical experiment with amiloride is described below.

### Methods

Animal Preparation: Adult ewes (ranging in weight from 25 to 35 kg) were restrained in an upright position in a specialized body harness adapted to a modified shopping cart. The animals' heads were immobilized and local anesthesia of the nasal passage was induced with 2% lidocaine. The animals were then nasally intubated with a 7.5 mm internal diameter endotracheal tube (ETT). The cuff of the ETT was placed just below the vocal cords and its position was verified with a flexible bronchoscope. After intubation the animals were allowed to equilibrate for approximately 20 minutes prior to initiating measurements of mucociliary clearance.

Administration of Radio-aerosol: Aerosols of ^{99m}Tc-Human serum albumin (3.1 mg/ml; containing approximately 20 mCi) were generated using a Raindrop Nebulizer which produces a droplet with a median aerodynamic diameter of 3.6 μm. The nebulizer was connected to a dosimetry system consisting of a solenoid valve and a source of compressed air (20 psi). The output of the nebulizer was directed into a plastic T connector; one end of which was connected to the endotracheal tube, the other was connected to a piston respirator. The system was activated for one second at the onset of the respirator's inspiratory cycle. The respirator was set at a tidal volume of 500 mL, an inspiratory to expiratory ratio of 1:1, and at a rate of 20 breaths per minute to maximize the central airway deposition. The sheep breathed the radio-labeled aerosol for 5 minutes. A gamma camera was used to measure the clearance of ^{99m}Tc-Human serum albumin from the airways. The camera was positioned above the animal's back with the sheep in a natural upright position supported in a cart so that the field of image was perpendicular to the animal's spinal cord. External radio-labeled markers were placed on the sheep to ensure proper alignment under the gamma camera. All images were stored in a computer integrated with the gamma camera. A region of interest was traced over the image corresponding to the right lung of the sheep and the counts were recorded. The counts were corrected for decay and expressed as percentage of radioactivity present in the initial baseline image. The left lung was excluded from the analysis because its outlines are superimposed over the stomach and counts can be swallowed and enter the stomach as radio-labeled mucus.

Treatment Protocol (Assessment of activity at t-zero): A baseline deposition image was obtained immediately after radio-aerosol administration. At time zero, after acquisition of the baseline image, vehicle control (distilled water), amiloride or other experimental compounds were aerosolized from a 4 ml volume using a Pari LC JetPlus nebulizer to free-breathing animals. The nebulizer was driven by compressed air with a flow of 8 liters per minute. The time to deliver the solution was 10 to 12 minutes. Animals were extubated immediately following delivery of the total dose in order to prevent false elevations in counts caused by aspiration of excess radio-tracer from the ETT. Serial images of the lung were obtained at 15-minute intervals during the first 2 hours after dosing and hourly for the next 6 hours after dosing for a total observation period of 8 hours. A washout period of at least 7 days separated dosing sessions with different experimental agents.

Treatment Protocol (Assessment of Activity at t-4 hours): The following variation of the standard protocol was used to assess the durability of response following a single exposure to vehicle control (distilled water), amiloride or benzamil or other investigational agents. At time zero, vehicle control (distilled water), positive control (amiloride), or investigational compounds were aerosolized from a 4 ml volume using a Pari LC JetPlus nebulizer to free-breathing animals. The nebulizer was driven by compressed air with a flow of 8 liters per minute. The time to deliver the solution was 10 to 12 minutes. Animals were restrained in an upright position in a specialized body harness for 4 hours. At the end of the 4-hour period animals received a single dose of aerosolized ^{99m}Tc-Human serum albumin (3.1 mg/ml; containing approximately 20 mCi) from a Raindrop Nebulizer. Animals were extubated immediately following delivery of the total dose of radio-tracer. A baseline deposition image was obtained immediately after radio-aerosol administration. Serial images of the lung were obtained at 15-minute intervals during the first 2 hours after administration of the radio-tracer (representing hours 4 through 6 after drug administration) and hourly for the next 2 hours after dosing for a total observation period of 4 hours. A washout period of at least 7 days separated dosing sessions with different experimental agents.

Statistics: Data were analyzed using SYSTAT for Windows, version 5. Data were analyzed using a two-way repeated ANOVA (to assess overall effects), followed by a paried t-test to identify differences between specific pairs. Significance was accepted when P was less than or equal to 0.05. Slope values (calculated from data collected during the initial 45 minutes after dosing in the t-zero assessment) for mean MCC curves were calculated using linear least square regression to assess differences in the initial rates during the rapid clearance phase.

While the invention has been described with reference to preferred aspects, it is to be understood that variations and modifications may be resorted to as will be apparent to those skilled in the art. Such variations and modifications are to be considered within the purview and the scope of the claims appended hereto.

1. A method of preventing, delaying, inhibiting, and/or reducing the risk of infection from airborne pathogens, comprising:
administering an effective amount of an active to prevent, delay, inhibit, and/or reduce the risk of infection from airborne pathogens, the active consisting essentially of amiloride, benzamil, phenamil or a pharmaceutically acceptable salt thereof to an individual in need of protection against infection or disease from one or more airborne pathogens.

2. The method of claim 1 wherein the pathogen is Bacillus anthracis.

3. The method of claim 1 wherein the pathogen is Variola major.

4. The method of claim 1 wherein the pathogen is Yersinia pestis.

5. The method of claim 1 wherein the pathogen is Francisella tularensis.

6. The method of claim 1 wherein the pathogen is a gram negative bacteria.

7. The method of claim 6 wherein the gram negative bacteria is selected from the group consisting of Brucella species, Burkholderia pseudomallei, Burkholderia mallei, Coxiella burnetii and Rickettsia.

8. The method of claim 1 wherein the pathogen is an alphavirus, a flavivirus or a bunyavirus.

9. The method of claim 1 wherein the pathogen is ricin toxin from Ricinus communis, epsilon toxin of Clostridium perfringens or Staphylococcal enterotoxin B.

10. The method of claim 1 wherein the pathogen is Mycobacterium tuberculosis bacteria.

11. The method of claim 1 wherein the pathogen is an influenza virus, rhinovirus, adenovirus or respiratory syncytial virus.

12. The method of claim 1 wherein the pathogen is coronavirus.

13. The method of claim 1 wherein the amiloride, benzamil, phenamil or pharmaceutically acceptable salt thereof is administered in an aerosol suspension of respirable particles which the individual inhales.

14. A method for reducing the risk of infection from an airborne pathogen which can cause a disease in a human, said method comprising administering an effective amount of an active to prevent, delay, inhibit, and/or reduce the risk of infection from the airborne pathogen in the human, the active consisting essentially of amiloride, benzamil, phenamil or a pharmaceutically acceptable salt thereof to the lungs of the human who may be at risk of infection from the airborne pathogen but is asymptomatic for the disease.

15. The method of claim 14 wherein the airborne pathogen is Bacillus anthracis and the disease is anthrax.

16. The method of claim 14 wherein the airborne pathogen is Variola major and the disease is small pox.

17. The method of claim 14 wherein the airborne pathogen is Yersinia pestis and the disease is plague.

18. The method of claim 14 wherein the airborne pathogen is a gram negative bacteria.

19. The method of claim 18 wherein the gram negative bacteria is selected from the group consisting of Brucella species, Burkholderia pseudomallei, Burkholderia mallei, and Coxiella burnetii.

20. The method of claim 14 wherein the airborne pathogen is an alphavirus, a flavivirus or a bunyavirus.

21. The method of claim 14 wherein the airborne pathogen is ricin toxin from Ricinus communis, epsilon toxin of Clostridium perfringens or Staphylococcal enterotoxin B.

22. The method of claim 14 wherein the airborne pathogen is Mycobacterium tuberculosis bacteria.

23. The method of claim 14 wherein the airborne pathogen is an influenza virus, rhinovirus, adenovirus or respiratory syncytial virus.

24. The method of claim 14 wherein the airborne pathogen is coronavirus and the disease is severe acute respiratory syndrome.

25. The method of claim 14 wherein the amiloride, benzamil, phenamil or pharmaceutically acceptable salt thereof is administered in an aerosol suspension of respirable particles which the human inhales.
